(19) United States
(12) Patent Application Publication
(10) Pub. No.: US 2026/0335501 A1
(43) Pub. Date: Oct. 29, 2026

(54) METHOD OF MANUFACTURING A SILVER DENDRITE PLATFORM FOR BIOMEDICAL APPLICATIONS

(71) Applicant: CONSIGLIO NAZIONALE DELLE RICERCHE, Roma (IT)

(72) Inventors: Sabrina CONOCI, Catania (IT); Alessia IRRERA, Messina (IT); Giovanna CALABRESE, Trecastagni (IT); Emanuele Luigi SCIUTO, Catania (IT); Antonio Alessio LEONARDI, Acireale (IT); Corrado Rosario SPINELLA, Catania (IT); Domenico FRANCO, Capri Leone (IT); Salvatore GUGLIELMINO, Catania (IT); Maria Giovanna RIZZO, Santa Flavia (IT)

(73) Assignee: CONSIGLIO NAZIONALE DELLE RICERCHE, Roma (IT)

(21) Appl. No.: 19/115,260

(22) PCT Filed: Oct. 2, 2023
(86) PCT No.: PCT/IB2023/059847

§ 371 Date: Mar. 26, 2025

(30) Foreign Application Priority Data
Sep. 30, 2022 (IT) 102022000020145

(51) Int. Cl. A61L 27/04; A61L 27/54; B82Y 5/00; B82Y 40/00; C09K 13/04; C30B 7/14; C30B 29/02; C30B 29/62

(52) U.S. Cl. CPC A61L 27/047; A61L 27/54; B82Y 5/00; B82Y 40/00; C09K 13/04; C30B 7/14; C30B 29/02; C30B 29/62; A61L 2300/104; A61L 2300/404; A61L 2400/12

(57) ABSTRACT

Method of manufacturing a silver dendrite platform for biomedical applications, comprising the steps of providing a silicon wafer (10) stripped of its native surface silicon oxide layer and immersing said silicon wafer in a solution of silver nitrate and hydrofluoric acid. In said step of immersing the silicon wafer in the solution of silver nitrate and hydrofluoric acid, the silver nitrate dissociates into NO₃⁻ anions and Ag⁺ cations, and said Ag⁺ cations then precipitate on the silicon wafer, thus resulting in the formation of multiple silver dendrites (25). The method further comprises the step of separating the silver dendrites from the silicon wafer thus obtaining a layer of isolated silver dendrites.

### CROSS REFERENCE TO RELATED APPLICATIONS AND INCORPORATION BY REFERENCE

This application claims priority from commonly owned PCT Patent Application PCT/IB2023/059847 filed 2 Oct. 2023 and titled “METHOD OF MANUFACTURING A SILVER DENDRITE PLATFORM FOR BIOMEDICAL APPLICATIONS”, and from commonly owned Italian Patent Application 102022000020145 filed 30 Sep. 2022 and titled “Method Of Manufacturing A Silver Dendrite Platform For Biomedical Applications”. This application also incorporates by this reference the entirety of PCT Patent Application PCT/IB2023/059847, and the entirety of Italian Patent Application 102022000020145.

### TECHNICAL FIELD

The present invention relates to a method of manufacturing a silver dendrite platform for biomedical applications as well as to the platform thus obtained. The invention further relates to a prosthetic device comprising said silver dendrite platform.

### Background Art

One of the most dreaded complications following the implantation of a prosthetic device is periprosthetic infection, as the metal surface of the implant is an ideal breeding ground for bacteria and the formation of a biofilm.

In Italy the rate of prosthetic infection is 2-5% and the cost for a revision of an infected prosthesis is 4.8 times that of a primary implant. In addition, periprosthetic infection causes bone-destroying processes that lead not only to the loosening of the prosthesis but also to the progressive destruction of the joint as a whole.

Among the most widely used systems to reduce biofilm formation in orthopaedic implants is the use of metals, including silver, copper, gold, titanium and zinc, in the preparation of implantable devices. At the nanoparticle scale, however, the most interesting metals seem to be copper and, above all, silver. In fact, silver nanoparticles have been widely studied in recent years because of their ability to reduce the adhesion of fungi and bacteria.

However, although silver nanoparticles have shown exceptional antibacterial and anti-biofilm properties, they present cytotoxicity problems that severely limit their use, especially in the biomedical field. Researchers are still trying to determine the optimal amount and size of silver nanoparticles to be used in implantable materials or for doping implantable materials in order to avoid possible adverse effects. Unfortunately, this is one of the most important limitations for the use of silver nanostructures in the manufacture of implantable orthopaedic devices to date.

The object of the invention is to overcome the problems and limitations of current prosthetic devices by providing a platform for biomedical applications in the orthopaedic field that exhibits exceptional antimicrobial capabilities while having good compatibility with eukaryotic cells.

This and other objects are achieved with the platform for biomedical applications obtained by the method as claimed in the appended claims.

### SUMMARY OF INVENTION

The method of manufacturing a silver dendrite platform according to the invention comprises the steps of:
providing a silicon wafer stripped of its native surface silicon oxide layer,
immersing said silicon wafer in a solution of silver nitrate and hydrofluoric acid.

In said step of immersing the silicon wafer in the solution of silver nitrate and hydrofluoric acid, the silver nitrate dissociates into NO₃⁻ anions and Ag⁺ cations, and said Ag⁺ cations then precipitate on the silicon wafer, thus resulting in the formation of multiple silver dendrites. Preferably, said step of immersing the silicon wafer in the solution of silver nitrate and hydrofluoric acid is carried out at room temperature and for essentially 30 minutes.

The method further comprises the step of separating the silver dendrites from the silicon wafer thus obtaining a layer of isolated silver dendrites, i.e., the silver dendrite platform.

According to the invention, in the step of immersing the silicon wafer in the solution of silver nitrate and hydrofluoric acid, a matrix of silicon nanowires forms on the surface of the silicon wafer by the process of metal-assisted chemical etching, wherein the silver formed on the surface of the wafer acts as catalyst for said chemical etching.

Advantageously, the silver dendrites of the platform obtained by the method according to the invention have a fractal-like structure.

The method makes it possible to obtain silver dendrites having a length between 50 nm and 20 μm and a thickness between 10 μm and 30 μm.

Advantageously, the silver dendrite platform obtained by the method according to the invention exhibits excellent antimicrobial capabilities and has shown good compatibility with eukaryotic cells, therefore being particularly suitable for biomedical applications, especially for prosthetic devices such as orthopaedic or dental implants.

The pronounced antimicrobial activity of the silver dendrites of the platform is also due to their enormous surface-to-volume ratio, due to their fractal-like structure, which allows the surface area available for antibacterial activity to be greatly increased for the same volume. Furthermore, it can be hypothesised that the absence of cytotoxicity in silver dendrites may be due to the absence or low presence of silver ions in the biological environment with which they are in contact (cell matrix, cells . . . ).

### BRIEF DESCRIPTION OF DRAWINGS

These and other features and advantages of the present invention will become evident from the following description of preferred embodiments given by way of non-limiting examples with reference to the annexed drawings, in which parts identified with identical or similar reference numerals denote parts having identical or similar function and construction, and in which:

FIGS. 1a-1b schematically represent an intermediate step and the final step of the method of manufacturing the platform according to an embodiment of the invention;

FIG. 2 shows an SEM cross-sectional image of silver dendrites, having an average thickness of 15±5 μm, on a matrix of silicon nanowires (Si NW) of about 2 μm in height, obtained by the method of manufacturing the platform according to an embodiment of the invention;

FIG. 3 shows an in-plane SEM image of silver dendrites obtained by the method of manufacturing the platform according to an embodiment of the invention;

FIG. 4 shows a prosthetic device (femoral prosthesis) comprising platforms of silver fractals according to the invention;

FIGS. 5a-5b show photos of the results of the antibacterial activity on bacterial cultures of S. aureus ATCC 29213 and P. aeruginosa ATCC 27853, in the absence (FIG. 5a) and presence (FIG. 5b) of silver dendrite platforms;

FIGS. 6a-6b show the graphs of vital count results, in CFU/ml (colony-forming units per millimetre), performed on bacterial cultures of S. aureus ATCC 29213 and P. aeruginosa ATCC 27853 in the absence and presence of silver dendrite platforms (FIG. 6a) and the bacterial viability compared to the cultures in the absence of the bacterial platforms (bacterial viability vs CTR) (FIG. 6b);

FIG. 7 shows the graph of the cell viability of eukaryotic cells (hFOB, human fetal osteoblasts) in the presence of silver dendrites as a percentage value compared to that in the absence of silver dendrites.

### DESCRIPTION OF EMBODIMENTS

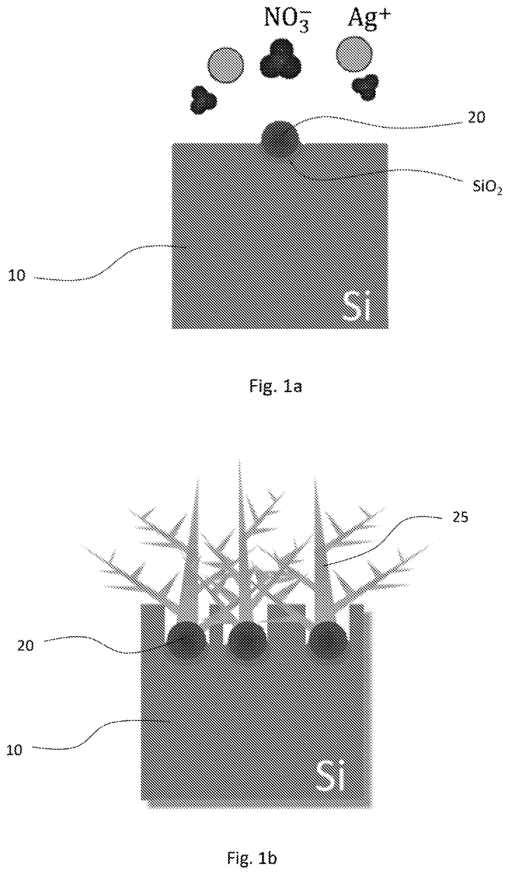

A method of manufacturing a silver dendrite platform for biomedical applications according to a preferred embodiment is described here below with reference to FIGS. 1a-b.

The platform is obtained on the surface of a silicon wafer 10. In particular, after an initial step of typical cleaning of the silicon wafer 10, for example by means of bathing in acetone for 5 minutes, in isopropanol for 5 minutes and rinsing with water, the silicon wafer 10 is preferably cleaned of organic contaminants by means of a UV-ozone treatment for 2 minutes. After cleaning, the native oxide covering the silicon wafer 10 is lifted off by using an aqueous solution of hydrofluoric acid 5%.

After said step, the wafer 10 is immersed in a solution containing 0.02 M silver nitrate (AgNO₃), as silver precursor, and 5 M hydrofluoric acid diluted in deionised water for 30 minutes at room temperature. In such solution, the silver salts dissociate into single NO₃⁻ anions and Ag⁺ cations, as shown in FIG. 1a, and said Ag⁺ cations precipitate as clusters on the silicon wafer. Thus, seeds of silver particles 20 form on the surface of the wafer, on which particles further Ag⁺ ions accumulate, thereby forming, as shown in FIG. 1b, silver dendrites 25, i.e., crystalline silver aggregates with an arborescent, fractal-like appearance.

During the same process, the silver particles also act as catalyst for the chemical etching of the underlying silicon, according to the process known as Metal-assisted Chemical Etching. This chemical etching causes, as a side product, the formation of a matrix of silicon nanowires (Si NW) 12 on the surface of the wafer 10.

The above-mentioned steps of formation of silver dendrites 25 and chemical etching are carried out at room temperature.

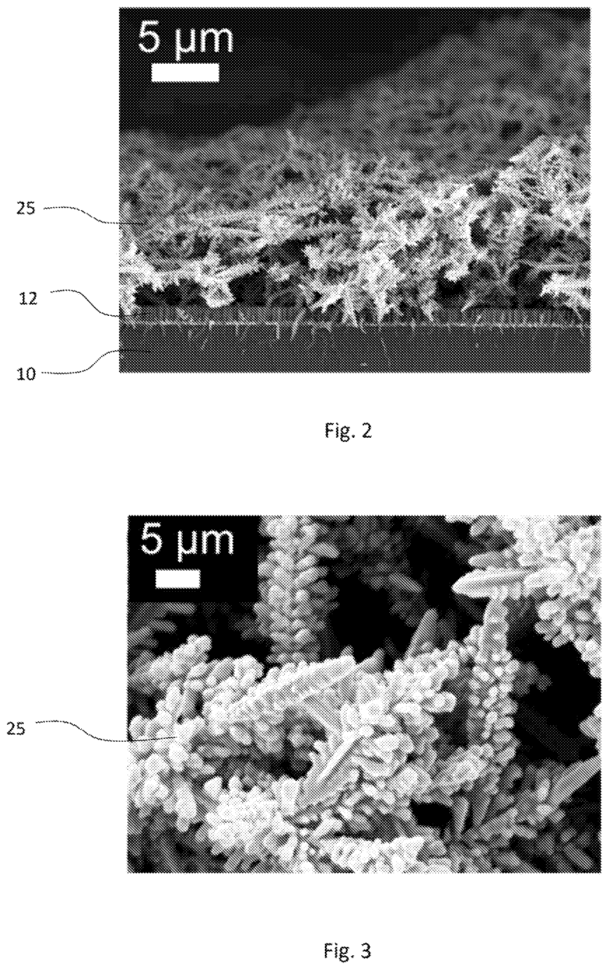

The silver dendrites 25 obtained by the method mentioned above have an average thickness of 15±5 μm and the silicon nanowires 12 on which the dendrites develop have a length of approximately 2 μm, as shown in the SEM images in FIGS. 2 and 3. The same images also show that the obtained silver dendrites 25 are organised in a dense forest.

According to other embodiments of the method of manufacturing the platform, by changing the duration of the chemical etching from 3 seconds to 30 minutes it is possible to obtain silver dendrites with different structural and morphological characteristics. For example, it is possible to obtain dendrites having a length between 50 nm e 20 μm and a thickness between 10 and 30 μm.

The silver dendrites 25 are then separated from the silicon wafer 10, thereby obtaining a layer of isolated silver dendrites, i.e., a silver dendrite platform 30. The platform 30 obtained according to the method described above can be used in multiple biomedical applications, particularly in prosthetic devices such as orthopaedic or dental implants.

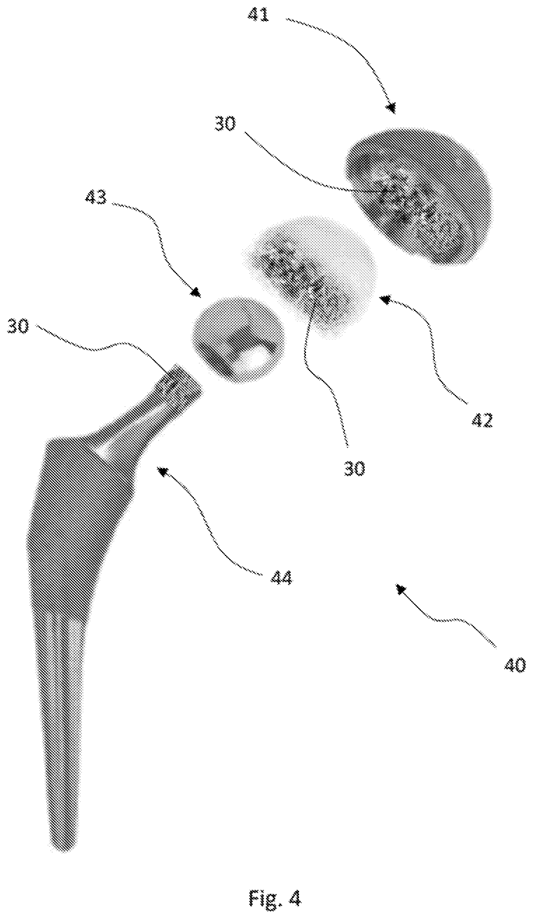

Referring to FIG. 4, an example of a prosthetic device comprising the silver dendrite platform is a femoral prosthesis 40. Said prosthesis 40 comprises an acetabular cup 41, usually made of titanium, an acetabular insert 42, usually made of plastics or ceramics, a femoral head 43, usually made of metal or ceramics, and a femoral stem 44, usually made of titanium. The silver dendrite platform 30 is applied between the acetabular cup 41 and the acetabular insert 42, between the acetabular insert 42 and the femoral head 43 and between the femoral head 43 and the femoral stem 44.

The silver dendrite platform exhibits excellent antimicrobial capabilities and good compatibility with eukaryotic cells, as evidenced by the results of microbiological and cellular analyses, some of which are reported below.

### Microbiological and Cellular Results

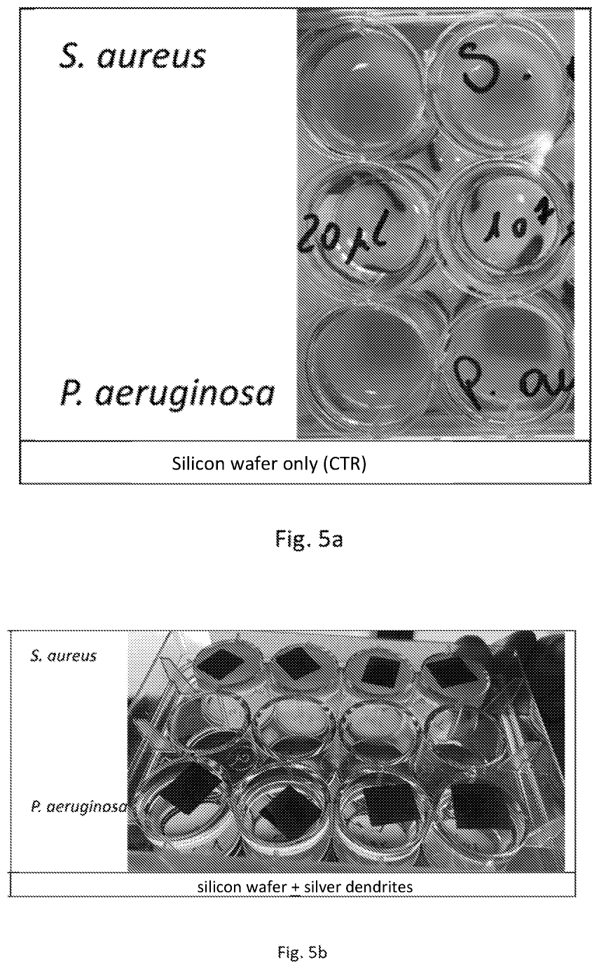

The results of the antibacterial activity on two different bacterial cultures, S. aureus ATCC 29213 (gram-positive) and P. aeruginosa ATCC 27853 (gram-negative), in the absence (FIG. 5a) and presence (FIG. 5b) of silver dendrite platforms, show that after 24 h incubation, only the P. aeruginosa culture, in the presence of silver dendrites, shows no bacterial growth, as indicated by the absence of turbidity in the culture medium. Conversely, the culture of S. aureus in the presence of silver dendrites shows bacterial growth, as indicated by the turbidity of the culture medium.

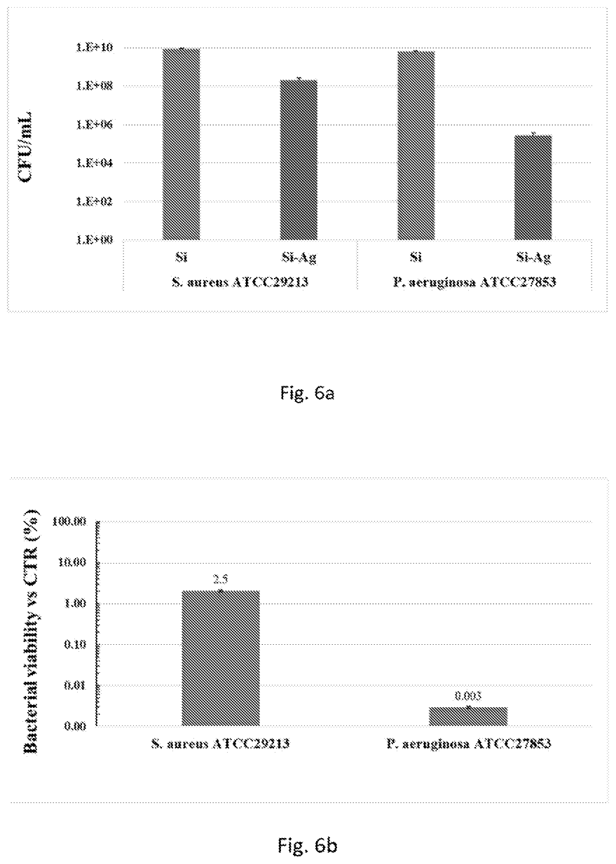

Furthermore, the viable count (CFU/mL) performed on the two different bacterial strains mentioned above indicated that S. aureus has a residual viability, in the presence of the silver dendrites, of 2.5% (a 97.5% reduction compared to the result without silver dendrites), whereas P. aeruginosa has a residual viability of 0.003% (a 99.997% reduction), as shown in the graphs in FIGS. 6a-b. The results therefore suggest a more effective antimicrobial activity of silver dendrites against the gram-negative bacterial strain.

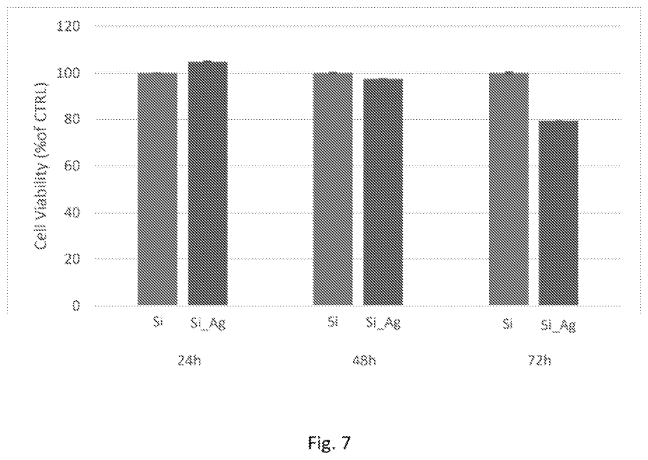

In addition, the data obtained from the assessment of the biocompatibility of eukariotic cells (hFOB, human fetal osteoblasts) in the presence of silver dendrites indicate the absence of cytotoxicity over time from 0 up to 72 hours. As shown in the graph in FIG. 7, in fact, the viability measured in the presence of silver dendrites is above the 70% threshold, which discriminates biocompatibility from potential cytotoxicity (according to the ISO 10993 standard).

1. Method of manufacturing a silver dendrite platform for biomedical applications, comprising the steps of:
providing a silicon wafer stripped of its native surface silicon oxide layer,
immersing said silicon wafer in a solution of silver nitrate and hydrofluoric acid, wherein, in said step of immersing the silicon wafer in the solution of silver nitrate and hydrofluoric acid, the silver nitrate dissociates into NO₃⁻ anions and Ag⁺ cations, and said Ag⁺ cations then precipitate on the silicon wafer, thus resulting in the formation of multiple silver dendrites,
separating the silver dendrites from the silicon wafer thus obtaining a layer of isolated silver dendrites.

2. Method according to claim 1, wherein the obtained silver dendrites have a fractal-like structure.

3. Method according to claim 1, wherein in said step of immersing the silicon wafer in the solution of silver nitrate and hydrofluoric acid, a matrix of silicon nanowires forms on the surface of the silicon wafer by the process of metal-assisted chemical etching, wherein the silver formed on the surface of the wafer acts as catalyst for said chemical etching.

4. Method according to claim 1, wherein said step of immersing the silicon wafer in the solution of silver nitrate and hydrofluoric acid is carried out substantially at room temperature.

5. Method according to claim 1, wherein said step of immersing the silicon wafer in the solution of silver nitrate and hydrofluoric acid is carried out for essentially 30 minutes.

6. Method according to claim 1, wherein the obtained silver dendrites have a length between 50 nm and 20 μm and a thickness between 10 μm and 30 μm.

7. Method according to claim 3, wherein the obtained silicon nanowires have a length of about 2 μm.

8. Prosthetic device comprising at least one silver dendrite platform obtained by the method according to claim 1.

9. Prosthetic device according to claim 8, said device being a femoral prosthesis comprising an acetabular cup, an acetabular insert, a femoral head and a femoral stem, wherein the silver dendrite platform is applied between the acetabular cup and the acetabular insert and/or between the acetabular insert and the femoral head and/or between the femoral head and the femoral stem.
